(19) United States
(12) Patent Application Publication
(10) Pub. No.: US 2024/0307195 A1
(43) Pub. Date: Sep. 19, 2024

(54) MONITORING LIMB DEPTH IN LOWER-LIMB PROSTHETIC SOCKETS

(71) Applicant: University of Washington, Seattle, WA (US)

(72) Inventors: Joan E. Sanders, Seattle, WA (US); Samuel Bennett, Seattle, WA (US); Clement Gurrey, Seattle, WA (US)

(73) Assignee: University of Washington, Seattle, WA (US)

(21) Appl. No.: 18/572,636

(22) PCT Filed: Jun. 21, 2022
(86) PCT No.: PCT/US2022/034396

§ 371 Date: Dec. 20, 2023

Related U.S. Application Data
(60) Provisional application No. 63/213,355, filed on Jun. 22, 2021.

(51) Int. Cl. A61F 2/80; A61F 2/76

(52) U.S. Cl. CPC A61F 2/80; A61F 2/76; A61F 2002/762

(57) ABSTRACT

In some embodiments, a sensor assembly for measuring a depth of insertion of a locking pin of a liner in a pin lock of a lower-limb prosthetic is provided. The sensor assembly comprises a base; a sensing element mounted to the base and having a hollow interior; and a housing that encloses at least the sensing element and includes means for coupling the sensor assembly to the lower-limb prosthetic in a position where the locking pin adapter of the lower-limb prosthetic is aligned with the hollow interior of the sensing clement. The sensing element is configured to generate a signal indicative of a size of a portion of the locking pin that is within the hollow interior of the sensing element.

### CROSS-REFERENCE TO RELATED APPLICATION

This application claims the benefit of Provisional Application No. 63/213,355, filed Jun. 22, 2021, the disclosure of which is hereby incorporated by reference herein in its entirety for all purposes.

### STATEMENT OF GOVERNMENT LICENSE RIGHTS

This invention was made with Government support under Contract No. W81XWH-16-C-0020 awarded by the US Army Medical Research Acquisition Activity (USAMRAA). The Government has certain rights in the invention.

### BACKGROUND

A typical lower-limb prosthesis is constructed from components that include a socket configured to accept the residual limb of the user, a pylon that supports the socket, and a foot that contacts the ground. An additional feature of lower-limb prostheses is a suspension system that helps retain the position of the residual limb within the socket of the prosthesis.

One important aspect of prosthesis design is socket fit. When designing a lower-limb prosthesis for a given user, the socket is shaped appropriately to support the residual limb such that the user's weight is distributed to reduce distal end bearing. When a prosthesis user's residual limb changes volume, socket fit may deteriorate. For example, when the residual limb loses volume, the residual limb may sink deeper into the socket and increase distal end bearing, putting inferior residual limb soft tissues at risk of injury. The residual limb may also experience greater motion along its longitudinal axis (cyclic superior-inferior motion, also referred to as “pistoning”) during walking. Particularly for inexperienced prosthesis users and people with insensate limbs, changes in limb position and motion may be difficult to perceive.

While some technologies have been developed to measure distal end bearing, limb distal position, and pistoning, these technologies often require additional hardware to be added to the interior of the socket to monitor the position of the residual limb. What is desired are technologies for monitoring residual limb position within a prosthetic socket without modifying other aspects of the prosthetic assembly.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a locking pin adapter for a lower-limb prosthetic is provided. The locking pin adapter comprises a pin lock body configured to receive a locking pin of a liner; a sensing element having a hollow interior and positioned to receive a portion of the locking pin that extends past the pin lock body; and sensing circuitry configured to generate a signal indicative of a size of the portion of the locking pin that is within the hollow interior of the sensing element.

In some embodiments, the sensing element includes a wire coil disposed around the hollow interior of the sensing element. In some embodiments, the circuitry includes a capacitor that forms an LC tank circuit with the coil of the sensing element. In some embodiments, the circuitry further includes a sensing circuit configured to measure an oscillation frequency of the LC tank circuit.

In some embodiments, the locking pin adapter further comprises data collection circuitry configured to receive the signal from the sensing circuitry and transmit collected data for storage on a computer-readable medium.

In some embodiments, the locking pin adapter further comprises a thermal sensor configured to generate a signal indicative of a temperature of the sensing element.

In some embodiments, a sensing system for measuring a depth of insertion of a locking pin of a liner in a pin lock of a lower-limb prosthetic is provided. The sensing system includes a sensor assembly that comprises a base; a sensing element mounted to the base and having a hollow interior; and a housing that encloses at least the sensing element and includes means for coupling the sensor assembly to the lower-limb prosthetic in a position where the locking pin adapter of the lower-limb prosthetic is aligned with the hollow interior of the sensing element. The sensing element is configured to generate a signal indicative of a size of a portion of the locking pin that is within the hollow interior of the sensing element.

In some embodiments, the sensing element includes a former that creates the hollow interior and a wire coil positioned around the former. In some embodiments, the sensing system further comprises a capacitor that forms an LC tank circuit with the wire coil. In some embodiments, the sensing system further comprises a sensing circuit configured to measure an oscillation frequency of the LC tank circuit, wherein the oscillation frequency is determined in part by the size of the portion of the locking pin that is within the hollow interior of the sensing element.

In some embodiments, the sensing element includes a thermal sensor configured to generate a signal indicative of a temperature of the sensing element.

In some embodiments, the sensor assembly further comprises a potting material within the housing.

In some embodiments, the means for coupling the sensor assembly to the lower-limb prosthetic includes a plurality of spacers positioned to transfer load from bolts of a socket of the lower-limb prosthetic to a pyramid adapter of the lower-limb prosthetic.

In some embodiments, the sensing system further comprises at least one spring positioned distally from the means for coupling the sensor assembly to the lower-limb prosthetic.

In some embodiments, a method of monitoring residual limb insertion depth in a socket of a lower-limb prosthetic is provided. A computing device receives values indicating an oscillation frequency of an LC tank circuit of a sensor assembly, wherein the oscillation frequency is affected in part by a size of a portion of a locking pin present within the sensor assembly after passing through a pin lock body of the lower-limb prosthetic. The computing system determines an insertion depth value based on the values indicating the oscillation frequency. The computing system stores the insertion depth value in a computer-readable medium.

In some embodiments, the method further comprises determining, by the computing system, a physical activity performed by a wearer of the lower-limb prosthetic based on insertion depth values stored in the computer-readable medium. In some embodiments, the determined physical activity includes at least one of weight shifting, standing, and walking.

In some embodiments, the method further comprises receiving, by the computing system, a signal indicating a temperature of the sensor assembly, and the insertion depth value is determined based further on the signal indicating the temperature.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a cross-sectional schematic diagram that illustrates a non-limiting example embodiment of a prosthesis configured to monitor residual limb depth within its socket according to various aspects of the present disclosure.

FIG. 2 is a cross-sectional schematic diagram that illustrates a portion of another non-limiting example embodiment of a prosthesis configured to monitor residual limb depth within its socket according to various aspects of the present disclosure.

FIG. 3 is a cutaway perspective illustration of a non-limiting example embodiment of a sensor assembly according to various aspects of the present disclosure.

FIG. 4 is a perspective illustration of a non-limiting example embodiment of a base assembly portion of a sensor assembly according to various aspects of the present disclosure.

FIG. 5 is a perspective illustration of a non-limiting example embodiment of a completed sensor assembly according to various aspects of the present disclosure.

FIG. 6 is a schematic illustration of the electronic design of a non-limiting example embodiment of a sensor assembly and a sensor controller according to various aspects of the present disclosure.

FIG. 7 is a perspective illustration of a non-limiting example embodiment of a calibration apparatus according to various aspects of the present disclosure.

FIG. 8 is a chart that illustrates calibration results of calibration of a non-limiting example embodiment of the sensor assembly according to various aspects of the present disclosure.

FIG. 9 is a chart that illustrates a typical sensed distance result from a study participant donning a prosthesis having the sensor assembly attached, weight shifting, and walking according to various aspects of the present disclosure.

FIG. 10 is a chart that illustrates results of tests with different panel radial distances.

FIG. 11 is a flowchart that illustrates a non-limiting example embodiment of a method of measuring residual limb insertion depth in a socket of a lower-limb prosthesis according to various aspects of the present disclosure.

FIG. 12 is a block diagram that illustrates a non-limiting example embodiment of a computing device appropriate for use as a computing device with embodiments of the present disclosure.

### DETAILED DESCRIPTION

One type of suspension is a locking pin suspension, in which a distal liner that is donned over the residual limb is connected to the socket using a pin and lock mechanism. Locking pin suspensions are one of the most commonly used means for suspension with lower-limb prostheses such as transtibial prostheses. Locking pin suspension systems allow for volume fluctuations, are trustworthy, do not require high dexterity, are usable by people with cognitive issues, and may include positive feedback such as audible clicks for security. Locking pin suspensions are considered simpler and easier to implement than alternatives such as sealing sleeves, suction, and elevated vacuum suspensions.

One common type of locking pin suspension uses a ratcheted pin and shuttle lock, although other types of locking pin suspensions are also available. In each type of locking pin suspension, the locking pin enters a pin lock body and is held in place by a pin lock mechanism. In order to provide adjustability, varying amounts of the locking pin pass into and through the pin lock body depending on how deeply the residual limb is situated within the socket.

Accordingly, the present disclosure describes techniques for monitoring limb depth within a prosthetic socket by monitoring a position of a locking pin. By measuring an amount of the locking pin that passes through the pin lock body and into a sensor assembly inserted between the pin lock body and the pylon or other distal assembly of the prosthesis, limb depth within the socket can be determined without alterations to the socket or other portions of the prosthesis.

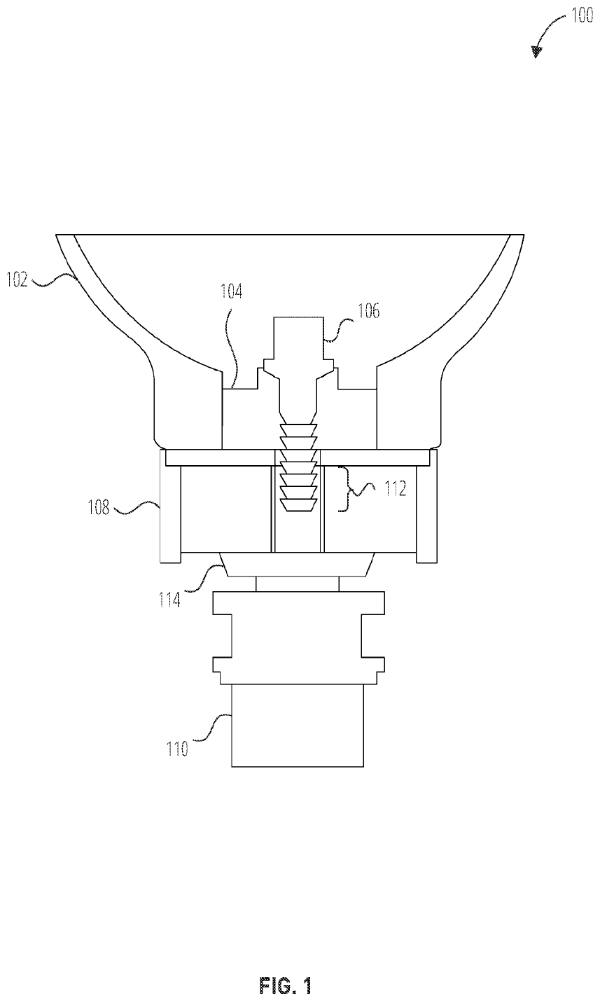

FIG. 1 is a cross-sectional schematic diagram that illustrates a portion of a non-limiting example embodiment of a prosthesis configured to monitor residual limb depth within its socket according to various aspects of the present disclosure. The prosthesis 100 may be a transtibial prosthesis, though in other embodiments, other types of lower-limb prostheses may be used, including transfemoral prostheses.

As shown, the prosthesis 100 includes a socket 102, a locking pin adapter 104, and a pylon 110. A locking pin 106 that is attached to an elastomeric liner worn over the residual limb (not shown) mates with the locking pin adapter 104 to provide suspension. The locking pin 106 is typically constructed from steel or another magnetically permeable material, or may be coated with a conductive material.

When the locking pin 106 is mated with the locking pin adapter 104, an extending portion 112 of the locking pin 106 extends distally beyond the locking pin adapter 104. The size of the extending portion 112 changes based on the limb depth within the socket 102. Accordingly, a sensor assembly 108 is positioned distally from the locking pin adapter 104 (between the locking pin adapter 104 and the pylon 110), such that the extending portion 112 of the locking pin 106 enters a hollow interior of a sensing element of the sensor assembly 108. The sensing element measures a size of the extending portion 112 and generates a signal that can be used to represent the limb depth within the socket 102.

Though the prosthesis 100 is illustrated with the sensor assembly 108 coupled directly between a locking pin adapter 104 and a pyramid adapter 114 attached to a pylon 110, in some embodiments, other prosthetic hardware known to those of skill in the art of prosthesis design may be present between the sensor assembly 108 and the pylon 110 or other distal prosthetic components. For example, in some embodiments, an alignment adapter, or another prosthetic component may be positioned immediately distally from the sensor assembly 108. Importantly, in some embodiments, the sensor assembly 108 may be added to the prosthesis 100 during assembly to provide limb depth monitoring functionality without modifying any of the other components of the prosthesis 100.

As illustrated, the locking pin 106 is a ratcheted pin for using with a shuttle lock-type locking pin adapter 104. The shuttle lock is distinctive in that as the residual limb settles into the socket 102, the locking pin 106 is retained by the locking pin adapter 104 at discrete locations as the locking pin 106 ratchets into the locking pin adapter 104. Once retained at a given position, the locking pin 106 may move distally as far as the next ratchet step, but may only move proximally to the given position. As illustrated below, the sensor assembly 108 is sensitive enough to detect not just the ratchet position to which the locking pin 106 has advanced, but also the tiny movements of the locking pin 106 between ratchet positions during activities such as walking and weight shifting. This high level of sensitivity may be useful not just in measuring socket fit, but also in measuring activity including but not limited to step counting, cadence, measurements of time standing versus sitting versus walking, measurements of continuous walking time, and analysis of weight shifting.

Though a ratcheted pin for use with a shuttle lock is primarily illustrated and described herein, this should not be seen as limiting. Any type of locking pin adapter and locking pin may be used wherein a portion of a magnetically permeable locking pin protrudes from the distal end of the locking pin adapter with sufficient length to enter the hollow interior of the sensor assembly 108. For example, another type of locking pin adapter 104 includes a gear that positively interfaces with teeth on the locking pin 106. As another example, another type of locking pin adapter 104 includes a clutch that grips a smooth locking pin 106. Still another example type of locking pin adapter 104 includes a cord or other flexible member that helps align a smooth locking pin 106 with the locking pin adapter 104 and passes through the locking pin adapter 104 when donned.

While an embodiment is illustrated in FIG. 1 wherein the sensor assembly 108 is a separate component of the prosthesis 100 that is attached under the locking pin adapter 104, this arrangement should not be seen as limiting. In some embodiments, components of the sensor assembly 108 (and particularly the sensing element 304, the thermal sensor 406, and the electrical connector 408) may be incorporated into the locking pin adapter 104 instead of being provided in a separate housing.

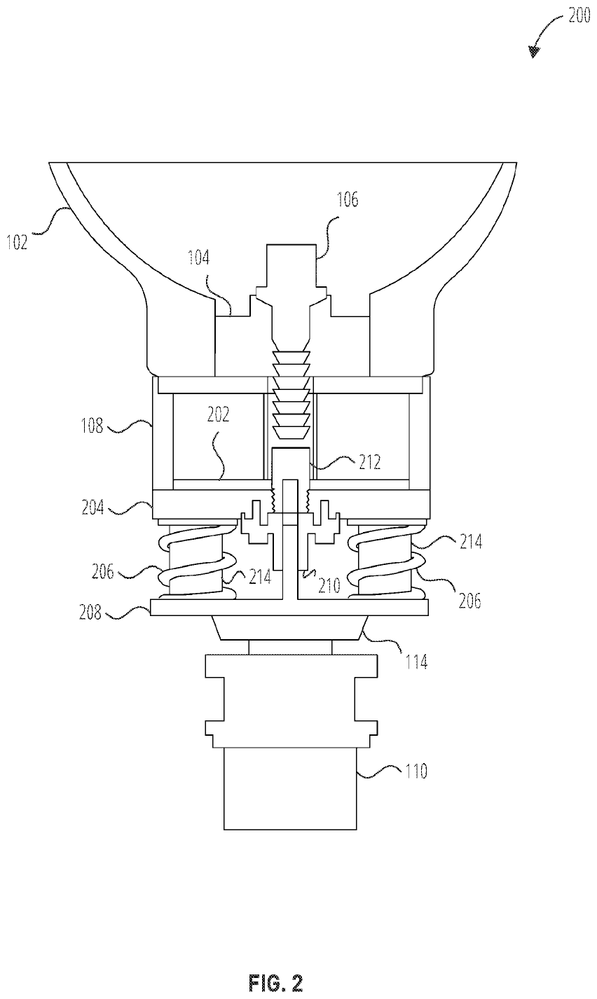

FIG. 2 is a cross-sectional schematic diagram that illustrates a portion of another non-limiting example embodiment of a prosthesis configured to monitor residual limb depth within its socket according to various aspects of the present disclosure.

Most of the components of the prosthesis 200 in FIG. 2 are the same as in FIG. 1, including the socket 102, the locking pin adapter 104, the locking pin 106, the sensor assembly 108, the pylon 110, and the pyramid adapter 114. The prosthesis 200 is different from the prosthesis 100 in that a spring or other resilient member may be positioned between the sensor assembly 108 pyramid adapter 114. In such embodiments, even if the locking pin 106 has bottomed out and is at a maximum depth within the locking pin adapter 104, the amount of the locking pin 106 that protrudes from the bottom of the locking pin adapter 104 and enters the sensor assembly 108 will change when different amounts of weight are borne by the socket 102 (e.g., while weight shifting or walking) and relevant data will continue to be collected.

As shown, instead of being coupled directly to the pyramid adapter 114, the sensor assembly 108 of prosthesis 200 sits atop a bearing plate 204. The bearing plate 204 includes passages for the spacers 214 of the sensor assembly 108 (discussed in further detail below), and the sensor assembly 108 moves axially along the spacers 214 when weight is applied to the socket 102. The spacers 214 are coupled to a spring plate 208, which is in turn coupled to the pyramid adapter 114. A set of springs 206 (or other resilient members) separates the bearing plate 204 from the spring plate 208. A linear bearing 210 restricts motion of the sensor assembly 108 to an axial direction in order to maintain the alignment between the locking pin 106 and the sensor assembly 108. A plunger 212 may also be present within the center of the bearing plate 204. One will recognize that although the discussion below refers primarily to the prosthesis 100, the discussion applies equally to the prosthesis 200, as the sensor assemblies 108 in the two prostheses are interchangeable and are simply mounted differently.

In FIG. 2, a nonconductive layer 202 is also illustrated on the bottom surface of the sensor assembly 108. The nonconductive layer 202 may be present in the sensor assembly 108 of the prosthesis 100 in FIG. 1, as well, and may have a thickness that is not illustrated to scale for the sake of clarity. The nonconductive layer 202 may be any suitable nonconductive material, such as ferrite or another material used to cover the wire coil 402 as discussed below. Presence of the nonconductive layer 202 may help isolate the sensing element 304 from unwanted interference from other components of the prosthesis 200, particularly components made of carbon fiber.

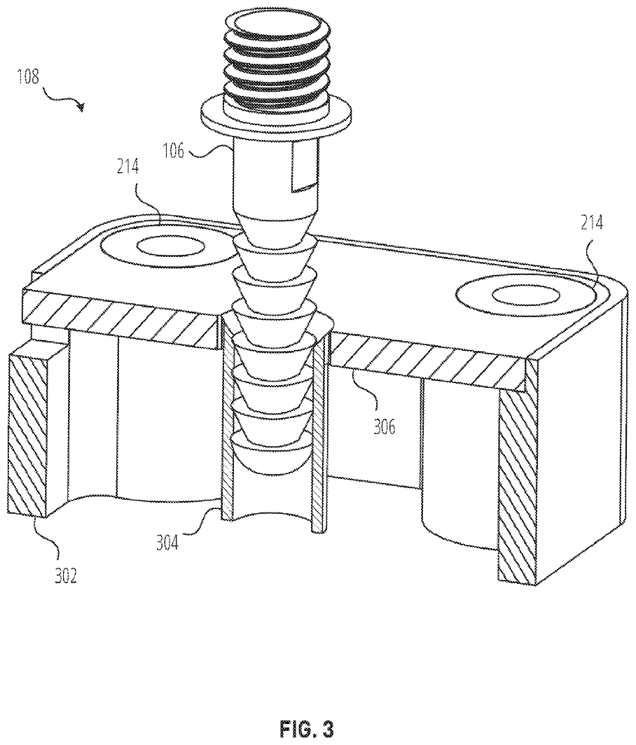

FIG. 3 is a cutaway perspective illustration of a non-limiting example embodiment of a sensor assembly according to various aspects of the present disclosure. The sensor assembly 108 of FIG. 3 is another view of the sensor assembly 108 illustrated as assembled into the prosthesis 100 in FIG. 1 and FIG. 2.

As shown, the sensor assembly 108 includes a housing 302, multiple spacers 214, a base 306, and a sensing element 304. The housing 302 may be constructed of any suitable material, and using any suitable technique. In some embodiments, the housing 302 may be printed using additive manufacturing techniques (e.g., 3D printing, etc.), subtractive manufacturing techniques (e.g., drilling, sculpting, CNC milling, etc.), injection molding techniques, or any other suitable technique. In some embodiments, the housing 302 may be 3D printed using a material such as VeroBlackPlus (RDG875), available from Stratasys.

In some embodiments, the housing 302 is shaped to hold the spacers 214 in an interference fit. In some embodiments, an additional detent or other structure may be used to retain the spacers 214 within voids of the housing 302 configured to receive the spacers 214. In some embodiments, an adhesive or other attachment means may be used to secure the spacers 214 within the housing 302. In some embodiments, the spacers 214 may be formed from aluminum or another suitable material. The spacers 214 transfer load from bolts in the socket 102 to the pylon 110 (or alignment adaptor, pyramid adaptor, or other distal structure of the prosthesis 100) to avoid loading the housing 302 or the sensing element 304. The housing 302 may be any appropriate size to contain the sensing element 304 and to couple with the other components of the prosthesis 100. In some embodiments, the housing 302 may have a length and width of 53.7 mm. When assembled, the entire sensor assembly may have a size of 53.7 mm×53.7 mm×20 mm, though other appropriate sizes may be used.

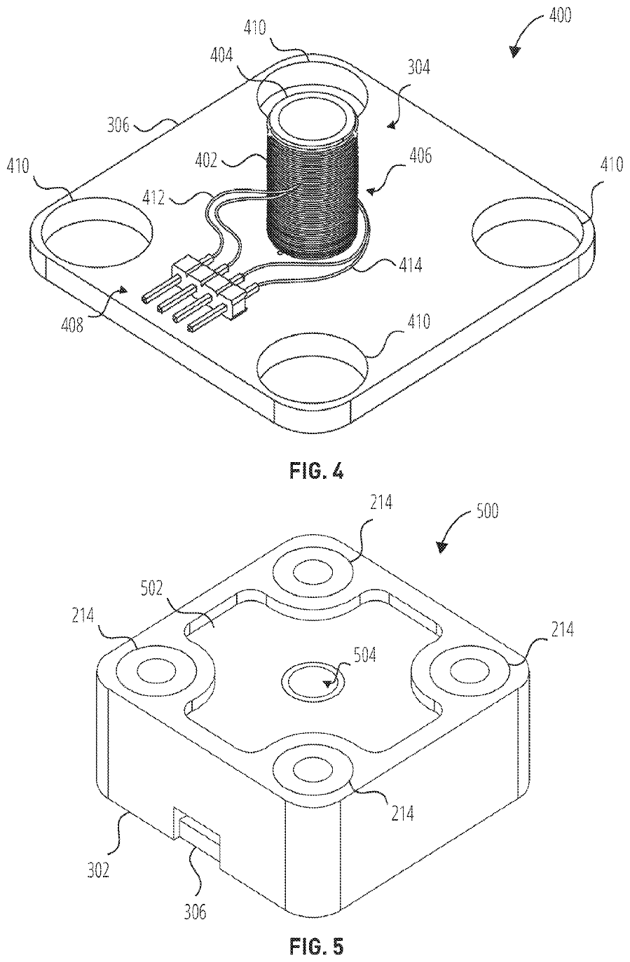

FIG. 4 is a perspective illustration of a non-limiting example embodiment of a base assembly portion of a sensor assembly according to various aspects of the present disclosure.

As shown, the base assembly 400 includes a base 306, an electrical connector 408, and a sensing element 304. In some embodiments, when the sensor assembly is assembled the housing 302 is placed atop the base 306, such that the base 306 and the housing 302 together form a reservoir for holding potting material, as discussed below. In some embodiments, after being filled with potting material, the base 306 and housing 302 may be inverted, such that the base 306 forms an upper portion of the sensor assembly 108 as illustrated in FIG. 3.

The base 306 includes holes 410 that align with the spacers 214 of the housing 302 and allow bolts of the socket 102 to pass through. In some embodiments, the holes 410 are sized to allow a portion of the spacers 214 that protrude from the housing 302 to pass through the holes 410 to allow the spacers 214 to directly bear load instead of loading the base 306. In some embodiments, the hole 410 may be sized to form an interference fit with the portions of the spacers 214 that protrude from the housing 302.

In some embodiments, the sensing element 304 includes an inductive sensor. In some embodiments, the inductive sensor includes a wire coil 402 wrapped around a former 404 that forms a hollow interior sized to accept the locking pin 106.

In some embodiments, the former 404 is made from a non-conductive material via any suitable technique, including but not limited to additive manufacturing, subtractive manufacturing, and injection molding. In some embodiments, the former 404 is 3D printed from a prototyping plastic such as VeroClear (RDG875), from Stratasys. One example of dimensions for the former 404 is an inner diameter of 8.00 mm to create a hollow interior to accommodate the locking pin 106 and an outer diameter of 10.16 mm, though other dimensions such as dimensions that are larger or smaller by up to 10% may be used. This 2.16 mm wall thickness may provide a sufficiently solid structure for winding the wire coil 402, though other thicknesses may be used. In some embodiments, the former 404 is affixed to the base 306 using adhesive, detents, or any other suitable attachment means. In some embodiments, the former 404 is positioned on the base 306 and is held in place with potting material. In some embodiments, the former 404 and the base 306 may be integrally formed, and the base 306 may be formed form a similar non-conductive material as the former 404.

In some embodiments, the wire coil 402 is made from copper wire. In some embodiments, the wire coil 402 is made from 30-AWG enameled copper wire (Remington Industries). In some embodiments, the wire coil 402 may be covered with a non-conductive material, such as ferrite, in order to isolate the wire coil 402 from unwanted influences. The wire coil 402 is electrically coupled to the electrical connector 408 via wires 412, and serves as an inductor (L) in a circuit with a capacitor (C, not shown) to form an LC tank circuit. One example capacitor that may be used is a 220 pF capacitor, though capacitors of other sizes may be used. The inductance of the wire coil 402 changes based on a size of a portion of the locking pin 106 that is present within the hollow interior of the former 404, thereby changing the oscillation frequency of the LC tank circuit and allowing the size of the portion of the locking pin 106 present within the hollow interior of the former 404 to be measured.

In some embodiments, a thermal sensor 406 (not visible, located behind the wire coil 402) is placed next to the wire coil 402 and is electrically coupled to the electrical connector 408 via wires 414. The thermal sensor 406 may be a thermistor or any other suitable type of temperature sensor configured to sense a temperature of the sensing element 304, in case the sensing element 304 is sufficiently sensitive to temperature that a thermal compensation strategy is desirable. In some embodiments, the thermal sensor 406 may be omitted.

While other types of sensing elements may be used (including but not limited to deflection sensors, optical sensors, linear encoders, and rotary encoders), the illustrated inductive sensor has benefits in that it does not require a line of sight, is lightweight to implement, and requires little power to operate.

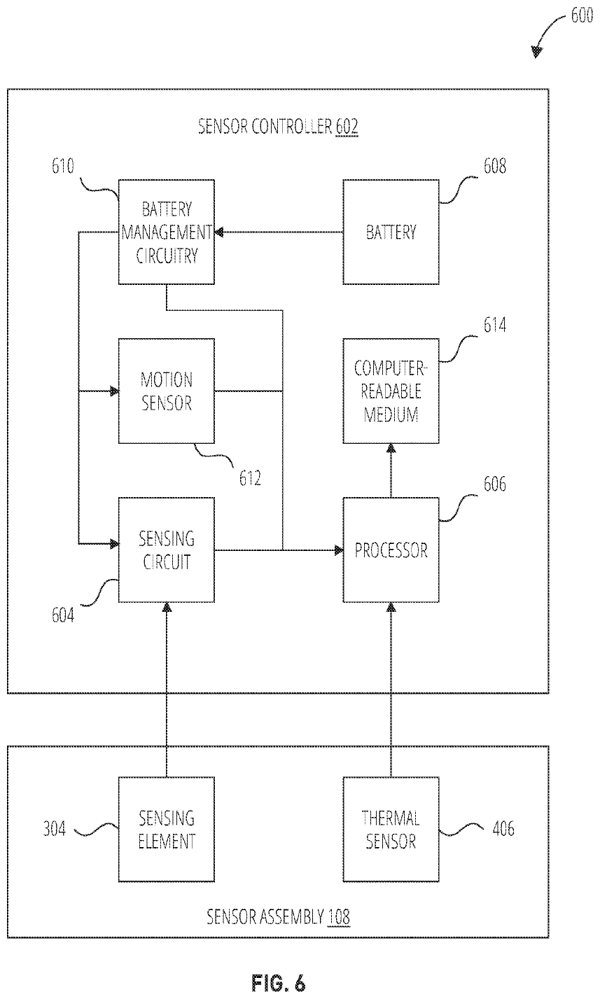

FIG. 5 is a perspective illustration of a non-limiting example embodiment of a completed sensor assembly according to various aspects of the present disclosure. As shown, the housing 302 has had the spacers 214 inserted therein and has been placed atop the base 306. A potting material 502 is inserted into the housing 302 around the components of the base 306, leaving a hollow interior 504 to accept the locking pin 106 and allowing it to pass into the sensing element 304. Any suitable potting material 502 may be used that secures the windings of the wire coil 402 on the former 404, strain relieves the wires 412 and the wires 414, and increases the rigidity of the completed sensor assembly 500. In some embodiments, a hard material (e.g., having a Shore A value in a range of 80-100, such as 90) may be used as the potting material 502. In some embodiments, a urethane rubber compound such as PMC-790, available from Smooth-On, may be used as the potting material 502.

FIG. 6 is a schematic illustration of the electronic design of a non-limiting example embodiment of a sensing system that includes a sensor assembly and a sensor controller according to various aspects of the present disclosure. As shown, the sensing system 600 comprises a sensor assembly 108 that is communicatively coupled to a sensor controller 602. In some embodiments, an electrical connection may be made using wires between the electrical connector 408 of the sensor assembly 108 and a corresponding electrical connector 408 of the sensor controller 602. The sensor controller 602 may be contained in a separate housing carried on an exterior surface of the prosthesis 100; on a belt, strap, or other carrying means to be carried by the user; or in any other suitable location.

As shown, the sensor controller 602 includes battery management circuitry 610, a motion sensor 612, a battery 608, a sensing circuit 604, and a processor 606. The battery management circuitry 610 includes circuitry for providing appropriate voltages from the battery 608 to the other components of the sensor controller 602 and sensor assembly 108. In some embodiments, the battery management circuitry 610 may include an on-board battery charger, voltage regulators, a buck converter, and a buck-boost converter to create power rails for the processor 606 and the sensing element 304. As a non-limiting example, the battery management circuitry 610 may include a BQ25895 battery charger from Texas Instruments, a TPS62740 buck converter from Texas Instruments, and a TPS630242 buck-boost converter from Texas Instruments.

In some embodiments, the battery 608 may be any suitable type of battery, including but not limited to a lithium-ion battery such as a 2000 mA-h lithium cobalt dioxide rechargeable battery. In some embodiments, the motion sensor 612 may be an accelerometer such as a 3-directional accelerometer. One non-limiting example of a suitable motion sensor 612 is a LSM6DS33 accelerometer from ST Microelectronics. The motion sensor 612 may be used to detect activity and activate/deactivate the components of the sensor controller 602 in order to conserve battery life. In some embodiments, the motion sensor 612 may be omitted.

In some embodiments, the sensing circuit 604 provides a voltage to the sensing element 304 via the electrical connector 408 and the wires 412 to induce oscillations in the LC tank circuit. The sensing circuit 604 is configured to measure the oscillation frequency of the LC tank circuit. In some embodiments, the output of the sensing circuit 604 in proximity counts is a ratio of the oscillation frequency of the LC tank circuit to an external clock frequency, which may be provided to the processor 606 via any suitable communication technique including but not limited to inter-integrated circuit (I2C) communication. One non-limiting example of a sensing circuit 604 includes a LDC1614 inductive sensing chip from Texas Instruments. In some embodiments, the sensor output may be provided in 28-bit resolution.

In some embodiments, the processor 606 receives the signal from the sensing circuit 604 and records received values in a computer-readable medium 614. In some embodiments, the processor 606 directly records the values received from the sensing circuit 604 in the computer-readable medium 614. In some embodiments, the processor 606 converts the values received from the sensing circuit 604 into measurements of insertion depth, and stores the insertion depth values in the computer-readable medium 614.

In some embodiments, the processor 606 is also communicatively coupled to the thermal sensor 406. The coupling between the processor 606 and the thermal sensor 406 may be by any suitable means. For example, voltages from the thermal sensor 406 may be processed by an analog-to-digital converter and provided digitally to the processor 606. As another example, voltages from the thermal sensor 406 may be provided to an analog input of the processor 606 and converted to digital values by analog-to-digital circuitry of the processor 606 itself.

In some embodiments, the computer-readable medium 614 may be a removable computer-readable medium, such as an SD card or other flash memory. The processor 606 may write the collected data to the computer-readable medium 614 via a serial peripheral interface (SPI). In some embodiments, the processor 606 may be a microcontroller. One non-limiting example of a suitable microcontroller for use as the processor 606 is a LPC54114 microcontroller from NXP Semiconductors. The processor 606 may sample all sensors (i.e., the sensing circuit 604 and optionally the thermal sensor 406 and/or the motion sensor 612) at a sampling rate such as 32.0 Hz+/−10%. An internal clock of the sensing circuit 604 may have a frequency between 35.0 MHz and 55.0 MHz, and is typically 43.4 MHz. Such an embodiment consumes about 2.0 mW of power during use and consumes about 52.8 μW during sleep mode.

Though the sensing system 600 is illustrated with particular components present in the sensor controller 602 and in the sensor assembly 108, one of ordinary skill in the art will recognize that the illustrated locations of components should not be seen as limiting, and that in other embodiments, some components may be moved between sensor controller 602 and the sensor assembly 108. For example, in some embodiments, some or all of the components illustrated as being in the sensor controller 602 may instead be present within the housing 302 of the sensor assembly 108. As another example, as mentioned elsewhere, some or all of the components of the sensing system 600 may be included as part of the locking pin adapter 104 instead of as a separate component.

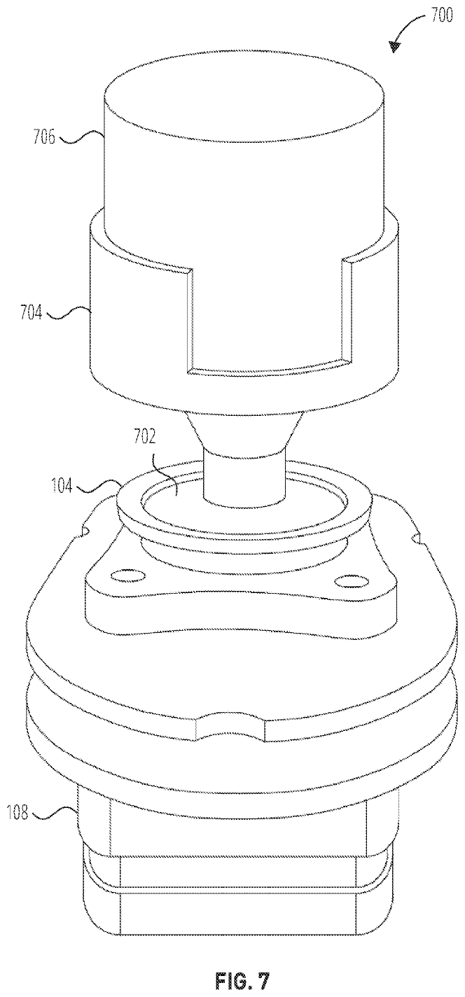

In some embodiments, the sensor assembly 108 may be calibrated using a benchtop jig that replaces the distal end of the socket 102. FIG. 7 is a perspective illustration of a non-limiting example embodiment of a calibration apparatus according to various aspects of the present disclosure.

As shown, the calibration apparatus 700 includes a locking pin adapter 104 that is separated from the other components of the prosthesis 100, and the sensor assembly 108 is coupled to the locking pin adapter 104 as illustrated in FIG. 1. Instead of the socket 102 being positioned proximal to the locking pin adapter 104, a weight holder 704 that has a locking pin 106 protruding from a distal portion is used. The weight holder 704 is separated from the locking pin adapter 104 by a replaceable calibration insert 702 of a known thickness. A weight 706 is placed in the weight holder 704 in order to simulate loading of the prosthesis 100.

In some embodiments, the calibration insert 702 is a 3D printed hollow cylinder made from a material such as VeroClear from Stratasys. In one non-limiting example, fourteen calibration inserts 702 of varying heights between 0.79 mm and 16.04 mm were placed one at a time within the calibration apparatus 700 in order to create different pin depths. Upon placing the calibration insert 702, a 1.0 kg weight 706 was placed in the weight holder 704, and measurements were collected at 32 Hz for 10 seconds. A mean of the central 250 samples is determined and used as the data point for that calibration insert 702. A dial indicator (such as a 2050SB-11 from Mitutoyo, having an accuracy of +/−0.01 mm for 0.00 up to 2.50 mm and +/−0.02 mm overall) may be used as a gold standard reference to measure the pin height.

The output of the sensing circuit 604 (in counts) may be plotted against pin height in millimeters, with 0.00 mm height being the pin at its maximum depth into the locking pin adapter 104. A calibration curve may then be derived from this information (e.g., a polynomial may be used for a least-squares fit), which may be used to convert clinically collected data from the sensing circuit 604 to pin height. In some embodiments, the data stored on the computer-readable medium 614 may be the output in cycles, and the conversion to pin height may be performed by a separate computing device. In some embodiments, the calibration curve may be stored on the processor 606, and the processor 606 may perform the conversion to pin height and store detected pin heights in millimeters in the computer-readable medium 614.

In some embodiments, the calibration apparatus 700 may also be used to evaluate performance characteristics of the sensor assembly 108 including drift and distance error from pin sagittal plane angulation, and effects of steel pin length and presence of a carbon fiber socket on calibration results. These tests are relevant because locking pin length and carbon fiber presence may vary from prosthesis to prosthesis. The repeatability of the calibration system was also tested. Mean root-mean-square (RMS) errors for sensor performance metrics were expressed in mm and percent full-scale output, and effects of variables on sensor calibration as a percentage change relative to that before the variable was changed.

Repeatability of the calibration system, also expressed as a percentage change, was evaluated by testing the same stainless-steel pin (34 mm, L-192003, Össur) three times and calculating the change in calibration from one trial to another (RMS error as a percentage of the mean). To calculate the effect of using a different pin, the difference in calibration between two pins of the same model was calculated and expressed as a percentage of the mean. Drift was tested by collecting data from the sensor overnight on three separate days using three different 34 mm length pins. Segments of data at least 1 h in duration where the temperature measured by the thermal sensor 406 stayed within ±0.04° C. were used. An RMS error was calculated and expressed in units of distance (mm). A ±0.04° C. range was arbitrarily selected. The impact of steel pin length was evaluated by calibrating a pin of length 44 mm and comparing the shape of the calibration curve with that from a 34 mm length pin. Presence of carbon fiber (CF) was tested by calibrating the sensor assembly 108 within an assembly similar to that shown in FIG. 7 but with a surrounding carbon fiber socket. A geometric model was used to investigate the pin height change from angulation within the former. Because the angle change was so small, the effect of pin misalignment with the former axis on the sensor's magnetic permeability was considered negligible.

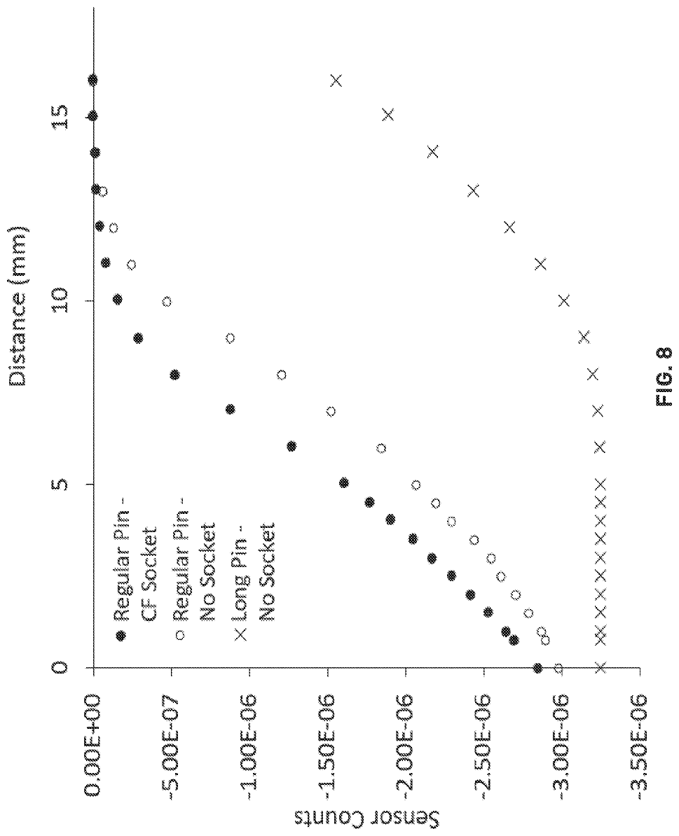

FIG. 8 is a chart that illustrates calibration results of calibration of a non-limiting example embodiment of the sensor assembly according to various aspects of the present disclosure. In the chart, sensor counts generated by the sensing circuit 604 for locking pins of different lengths and different heights relative to the locking pin adapter are shown. A distance of 0.00 mm is the maximum depth of a locking pin within the locking pin adapter. Results are shown for a standard locking pin of 34 mm length in a carbon fiber socket 102, the same locking pin without a socket 102, and a longer locking pin of 44 mm length without a socket 102.

The calibration results from the tests conducted on the two 34 mm pins (no socket) showed that calibration changed with distance. Mean sensitivity for the 0.00 to 4.49 mm distance (locking pin flush with the top of the housing 302 (0.00 mm) to 4.49 mm above the locking pin adapter 104) ranged from 4.601E5 to 4.844E5 counts/mm. For a 5.02 to 10.02 mm distance, it ranged from 6.751E5 to 7.147E5 counts/mm. For a 11.02 to 16.02 mm distance, it ranged from 8.620E4 to 9.005E4 counts/mm. In participant testing, the usable full-scale range was found to be 0.00 to 10.02 mm. Thus, only the first two ranges listed above were of clinical interest. A 3^{rd} order polynomial fit this portion of the calibration curve with a mean root mean square (RMS) error of 1.04% of the 10.02 mm full-scale output.

In bench testing, drift introduced less than a 0.001 mm/h error, and angulation of the pin within the former (pin sagittal plane rotation) introduced at most a 0.019 mm RMS error, as noted in the following table:

| | Mean RMS Error in Pin | | Maximum |
|---|---|---|---|
| | Height Measurement | | Full-Scale |
| | 0.00-4.49 mm | 5.02-10.02 | Output |
| Error Source | Range | mm Range | Error |
| Drift (Δt = 1 h) | <0.001 | <0.001 | <0.01% |
| Pin Angulation | 0.016 | 0.019 | 0.19% |
| Thermal (ΔT = 15° C.) | 0.017 | NA | 0.17% |
| (uncorrected) | | | |
| Thermal (ΔT = 15° C.) | <0.010 | NA | <0.10% |
| (corrected) | | | |

Using data from amputee participant testing, sensitivity to temperature was −1.137E−3 mm/° C. for the 0.00 mm to 4.49 mm distance range. Thus a 15° C, temperature change, which was the approximate temperature difference between body temperature (37° C.) and room temperature (22°° C.), introduced an error of 0.017 mm.

Field testing results demonstrated that temperature change had a measurable impact on sensor performance. Plots of both thermistor and sensor data over time showed that the shape of the thermistor signal and the pin sensor data during sitting were similar, indicating presence of a thermal dependence. A thermal sensitivity was determined by isolating sensor counts where the participant was still and the locking pin was fully seated in the locking pin adapter. For the isolated data, the sensor data from the sensing circuit 604 was plotted against the temperature data as measured by the thermal sensor 406 and a least squares linear fit was determined. All r values were above 0.97, and the mean sensitivity was determined to be −0.001142 mm/° C. (range −0.00054 to −0.00179 mm/° C.). When thermal compensation using this data was included, the error reduced to less than 0.010 mm. The RMS error for all three sources of error (drift, pin angulation, thermal (corrected)) was 0.21% of the 10-mm full-scale output.

The calibration apparatus 700 showed good repeatability in that calibration results were within 1.00% for both distance ranges (0.00 to 4.49 mm; 5.02 to 10.02 mm) when the same locking pin 106 was tested repeatedly, as shown in the following table. Using a different locking pin of the same model resulted in a 4.00% and 4.38% sensitivity change depending on the distance range. A longer locking pin (44 mm) shifted the calibration curve such that the locking pin produced little to no signal change for the 0.00 to 7.00 mm range, but a higher signal between 7.00 and 16.02 mm, as shown in FIG. 8. When a carbon fiber socket 102 was introduced, sensitivity increased 20.50% for the 0.00 to 4.49 mm range and decreased 16.60% for the 5.02 to 10.02 mm range.

| | Pin Height (0.00 mm is maximum | |
|---|---|---|
| | depth into the locking pin adapter) | |
| Variable | 0.00-4.49 mm | 5.02-10.02 mm |
| Repeatability (same locking | 0.45% | 0.73% |
| pin) | | |
| Different locking pin (same | 4.00% | 4.38% |
| model) | | |
| Carbon fiber socket presence | 20.50% | −16.60% |

In addition to the calibration and repeatability testing described above, participant testing of a non-limiting example embodiment of the sensor assembly was performed. Institutional review board approval (protocol #49624) for human subject testing and informed consent from the participant were obtained before any study procedures were initiated. Participants were included in testing if they were at least 18 years of age, had a transtibial amputation at least 18 months prior, and were using a definitive prosthesis with locking pin suspension that enabled them to walk at a self-selected speed for multiple 60 s bouts on a treadmill separated by 5 s stands. Exclusion criteria were use of an assistive aid (e.g., cane) and presence of skin breakdown. An adjustable socket 102 was used so that the effects of socket looseness on sensor data could be tested. The locking pin adapter 104 was a shuttle lock, and specifically was an Össur Icelock 700 series. A socket 102 that had three radially adjustable panels (anterior medial, anterior lateral, posterior midline) with one motor and frame per panel, was implemented in participant testing. A mobile phone app that controlled the motors presented the radial position of the panels and allowed the user to adjust the position. The sensor controller 602 was set at a sampling rate of 32 Hz for this study. A different sampling rate could be used if desired.

Initial tests showed that for some participants, walking with an enlarged socket 102 caused the residual limb to bottom out against the distal socket 102. In these cases, the sensor assembly 108 registered minimal change in distance upon greater distal end bearing. We suspected this happened because the urethane umbrella covering the distal end of the liner was relatively stiff and did not deform much upon distal end bearing. To increase the sensitivity of the sensor assembly 108 under these conditions, we affixed a ring of compressible material (6.5 cm outer diameter, 1.8 cm inner diameter) to the distal socket 102 using double backed tape (SpeedTape, FastCap). The ring was made of a prosthetic elastomer liner material (Alpha Silicone, Willow Wood) of thickness 3.8 mm and compressive stiffness 349 kPa. The material was expected to thin with greater weight bearing, and thus the signal generated by the sensor assembly 108 should increase with greater compression. After completion of the participant study described below, we conducted a series of investigations placing flat disks of putty between the umbrella and socket 102 and then applying stress to simulate walking so as to better understand where load was transferred from the umbrella to the socket 102.

In the clinical study, we recorded data from the outset of the session while the participant donned the socket 102 and weight-shifted to settle in. Then the participant walked on a treadmill, maintaining a self-selected walking speed. After an initial fitting of approximately 5 min, during which the participant and prosthetist used the phone app that controlled panel radial position to establish a comfortable socket size, the participant walked continuously for a total of 240 s, 60 s at each of four socket panel positions. The panel positions were measured by the motor's encoder on each panel and were set to four different positions using the mobile phone: self-selected and 1.00, 2.00, and 3.00 mm looser than the self-selected setting.

Collected data were converted to distance using the calibration data, and thermal compensation was executed as described above. The mean peak-to-peak distance change in pin distance for steps at each panel setting (each 60 s walk) was calculated. Data from one of the four walks was not normally distributed thus a Wilcoxon signed rank test was used for all comparisons.

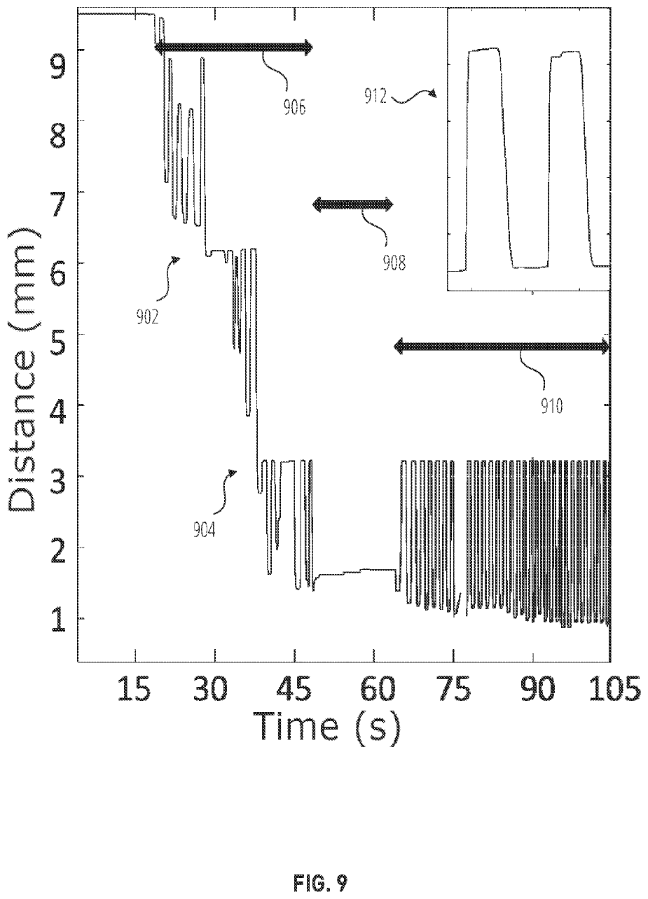

FIG. 9 is a chart that illustrates a typical sensed distance result from a study participant donning a prosthesis having the sensor assembly attached, weight shifting, and walking according to various aspects of the present disclosure. The data collected during donning (indicated by time span 906) show that the participant weight shifts approximately 11 cycles total to drive the locking pin 106 deep into the locking pin adapter 104. The locking pin 106 ratchets to deeper positions in the sixth cycle (point 902) and the eleventh cycle (point 904). The participant continues weight shifting for a few more cycles, stands for approximately fifteen seconds (time span 908), and then begins to walk continuously (time span 910). The participant walks at a self-selected walking speed of 1.7 mph. During swing phase, the locking pin 106 displaces proximally until caught on the ratchet (at 3.2 mm in FIG. 9), preventing further proximal displacement. During stance phase, the locking pin 106 displaces further distally with each step walked until reaching an approximately constant minimum at approximately 95 seconds. There is a brief data interruption (data acquisition error) illustrated at 77 seconds. The inset 912 shows a close-up view of the data for several step cycles.

Tests placing the putty in the distal socket showed that the putty tended to compress at the outer edge of the umbrella near the cylindrical part of the socket wall. This result was contrary to our expectation that the more central part of the umbrella provided support. Results from the participant walking on the treadmill with the elastomer ring between the umbrella and distal socket demonstrated an increase in peak-to-peak distance when the socket was enlarged from the self-selected panel position.

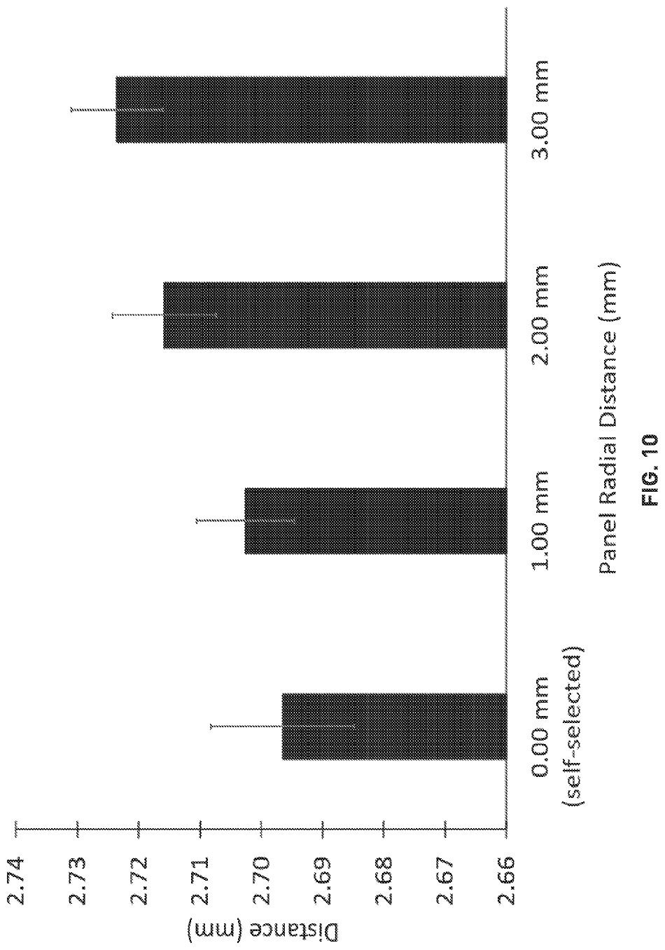

FIG. 10 is a chart that illustrates results of tests with different panel radial distances. Because the notch on the locking pin restricted proximal motion for all steps, the greater peak-to-peak distance was due to changes in the stance phase minima, i.e., locking pin depth into the locking pin adapter 104. Differences between all six pairs among the four settings were statistically significant (all p values<0.05). The magnitude of distance change across the entire range of adjustment was less than 0.30 mm.

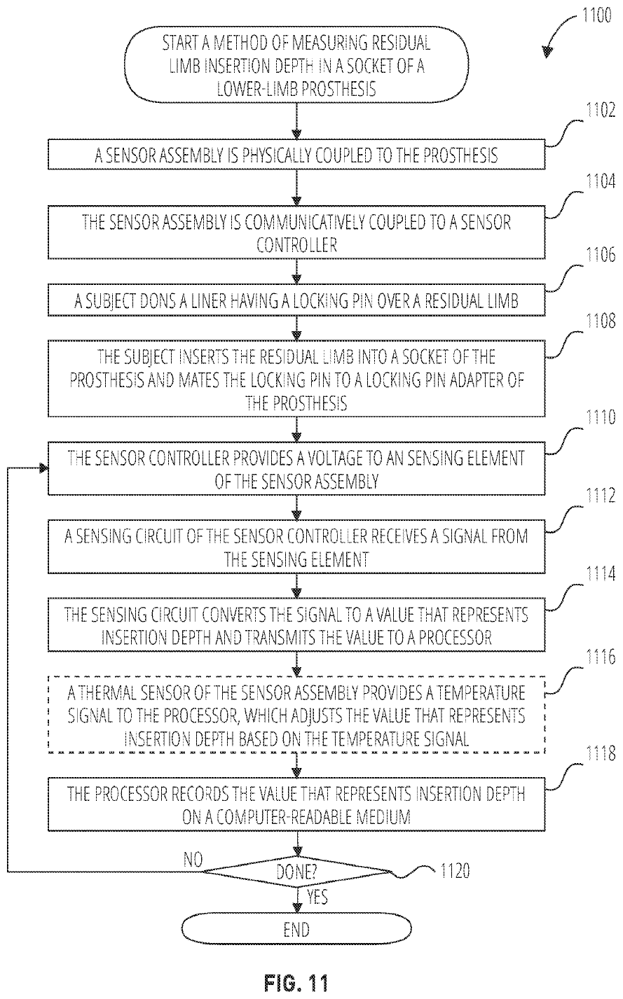

FIG. 11 is a flowchart that illustrates a non-limiting example embodiment of a method of measuring residual limb insertion depth in a socket of a lower-limb prosthesis according to various aspects of the present disclosure. The method 1100 illustrates a non-limiting example embodiment of the sensing techniques described above, and uses the sensing sensor assembly 108 and sensor controller 602 described above.

From a start block, the method 1100 proceeds to block 1102, where a sensor assembly 108 is physically coupled to the prosthesis 100. In some embodiments, the sensor assembly 108 is included as one of the components while assembling the prosthesis 100, and is included in the assembly distal to the locking pin adapter 104 so that a portion of the locking pin 106 that extends distally from the pin lock body enters the hollow interior 504 of the sensor assembly 108.

At block 1104, the sensor assembly 108 is communicatively coupled to a sensor controller 602. In some embodiments, the communicative coupling may include attaching a wire from the electrical connector 408 of the sensor assembly 108 to a connector of the sensor controller 602. In some embodiments, the communicative coupling may include conducting a wireless pairing between the sensor assembly 108 and the sensor controller 602.

At block 1106, a subject dons a liner having a locking pin 106 over a residual limb. At block 1108, the subject inserts the residual limb into a socket 102 of the prosthesis 100 and mates the locking pin to a locking pin adapter 104 of the prosthesis 100. As described above, when the locking pin is mated to the locking pin adapter 104, a portion of the locking pin extends distally through the pin lock body and enters the hollow interior 504 of the sensor assembly 108.

At block 1110, the sensor controller 602 provides a voltage to a sensing element 304 of the sensor assembly 108, and at block 1112, a sensing circuit 604 of the sensor controller 602 receives a signal from the sensing element 304. In embodiments wherein the sensing element 304 includes a wire coil 402 as an inductor of an LC tank circuit, the voltage applied to the wire coil 402 causes oscillations in the LC tank circuit, and the sensing circuit 604 detects the oscillations in the LC tank circuit. The rate of oscillations depends in part on the inductance of the wire coil 402, which in turn depends on the size of the portion of the locking pin that is present within the wire coil 402. In other words, the inductance of the sensing element 304 will change based on the size of the portion of the locking pin that protrudes distally from the locking pin adapter 104.

At block 1114, the sensing circuit 604 converts the signal to a value that represents insertion depth and transmits the value to a processor 606. In some embodiments, the sensing circuit 604 determines a count of oscillations of the LC tank circuit over a given time period, and provides the count of oscillations to the processor 606 as the value that represents insertion depth.

At optional block 1116, a thermal sensor 406 of the sensor assembly 108 provides a temperature signal to the processor 606, which adjusts the value that represents insertion depth based on the temperature signal. As described above, the processor 606 may use a calibration curve that corrects the value that represents insertion depth based on the temperature signal. The actions of optional block 1116 are described as optional because in some embodiments of the method 1100, temperature compensation is not used. Further, in some embodiments of the method 1100, the temperature signals may be collected, but may be stored by the processor 606 without correcting the value that represents insertion depth in order to allow temperature correction to be applied during post-processing and save computing resources at the sensor controller 602.

At block 1118, the processor 606 records the value that represents insertion depth on a computer-readable medium 614. In embodiments where the temperature signal is received as well, the processor 606 may also record the temperature signal on the computer-readable medium 614.

The method 1100 then proceeds to decision block 1120, where a determination is made regarding whether further monitoring should take place, or whether the method 1100 is done. If further monitoring should take place (e.g., because a test for the subject is still ongoing), then the result of decision block 1120 is NO, and the method 1100 returns to block 1110 to receive subsequent values from the sensor assembly 108. In some embodiments, the method 1100 may iterate through block 1110-block 1118 at a suitable sampling rate, such as 32 Hz+/−10%. In some embodiments, the method 1100 may not perform each block at each iteration. For example, the processor 606 may gather multiple values before recording them in a batch on the computer-readable medium 614 at block 1118.

Returning to decision block 1120, if further monitoring is not desired, then the result of decision block 1120 is YES, and the method 1100 proceeds to an end block, where it terminates. The gathered data stored on the computer-readable medium 614 may then be used for any suitable purpose, including but not limited to evaluating socket fit, step counting, analyzing cadence, measuring time standing versus sitting versus walking, measuring continuous walking time, and analyzing weight shifting behavior.

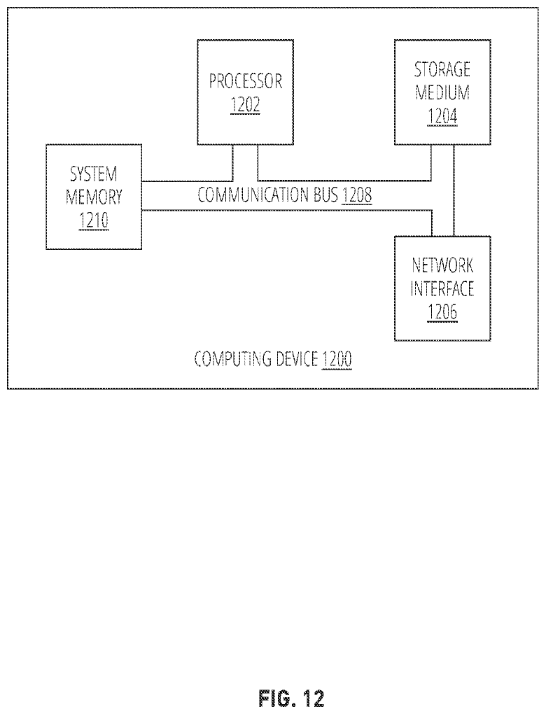

FIG. 12 is a block diagram that illustrates aspects of an exemplary computing device appropriate for use as a computing device of the present disclosure. The computing device 1200 may receive the values stored on the computer-readable medium 614 and perform the further processing for analyzing the gathered data as discussed above.

The example computing device 1200 describes various elements that are common to many different types of computing devices. While FIG. 12 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 1200 may be any one of any number of currently available or yet to be developed devices.

In its most basic configuration, the computing device 1200 includes at least one processor 1202 and a system memory 1210 connected by a communication bus 1208. Depending on the exact configuration and type of device, the system memory 1210 may be volatile or nonvolatile memory, such as read only memory (“ROM”), random access memory (“RAM”), EEPROM, flash memory, or similar memory technology. Those of ordinary skill in the art and others will recognize that system memory 1210 typically stores data and/or program modules that are immediately accessible to and/or currently being operated on by the processor 1202. In this regard, the processor 1202 may serve as a computational center of the computing device 1200 by supporting the execution of instructions.

As further illustrated in FIG. 12, the computing device 1200 may include a network interface 1206 comprising one or more components for communicating with other devices over a network. Embodiments of the present disclosure may access basic services that utilize the network interface 1206 to perform communications using common network protocols. The network interface 1206 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 1206 illustrated in FIG. 12 may represent one or more wireless interfaces or physical communication interfaces described and illustrated above with respect to particular components of the computing device 1200.

In the exemplary embodiment depicted in FIG. 12, the computing device 1200 also includes a storage medium 1204. However, services may be accessed using a computing device that does not include means for persisting data to a local storage medium. Therefore, the storage medium 1204 depicted in FIG. 12 is represented with a dashed line to indicate that the storage medium 1204 is optional. In any event, the storage medium 1204 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 1202, system memory 1210, communication bus 1208, storage medium 1204, and network interface 1206 are known and commercially available. For ease of illustration and because it is not important for an understanding of the claimed subject matter, FIG. 12 does not show some of the typical components of many computing devices. In this regard, the computing device 1200 may include input devices, such as a keyboard, keypad, mouse, microphone, touch input device, touch screen, tablet, and/or the like. Such input devices may be coupled to the computing device 1200 by wired or wireless connections including RF, infrared, serial, parallel, Bluetooth, Bluetooth low energy, USB, or other suitable connections protocols using wireless or physical connections. Similarly, the computing device 1200 may also include output devices such as a display, speakers, printer, etc. Since these devices are well known in the art, they are not illustrated or described further herein.

The sensor assembly 108 disclosed herein has potential application as a tool for patient training, diagnostic, and prognostic purposes. It quantifies a socket fit characteristic that prosthetists and patients cannot see-locking pin depth into the socket, which reflects residual limb distal position. Results from this study demonstrate that the sensor assembly 108 may have sufficient accuracy for identification of a loose socket. The sensor assembly 108 has potential application as a training tool to teach new prosthesis users how different limb depths feel, a remote monitor to provide diagnostic information for socket re-design or patient education, and a sensor for feedback control of automated systems intended to adjust socket size to maintain a specified limb depth.

The increase in peak-to-peak distance for an increase in socket size demonstrated in participant testing is consistent with clinical expectation-an increase in socket size caused greater residual limb depth into the socket, possibly increasing distal pressure. Statistically significant differences were observed between all pairs among the four settings. A reason for the low magnitude of pin depth change, less than 0.030 mm across the range of socket size adjustment, is that the elastomeric ring between the umbrella and socket, made of an essentially incompressible material, was under confined compression thus limiting its strain during walking. A person who did not start with distal contact would likely have a larger range of pin sensor displacement.

The change in sensitivity from using a different locking pin of the same model or a CF socket was greater than the repeatability error of the calibration jig, indicating that the sensor should be calibrated with the locking pin and socket with which it will be used. If the user's liner were to be replaced, recalibration would not be necessary, provided the same locking pin was used. Positioning the sensor in a carbon fiber socket shifted the calibration curve because the carbon fiber introduced a magnetically permeable material near the inductive sensor. The conductive properties of the carbon fiber did not play a meaningful role, presumably because the carbon fiber was a mesh, not a continuous material. Sensitivity was reduced for a long locking pin because the amount of steel material within the sensing element (the inductive coil) was increased. For participants whose socket causes distal end bearing and the sensor assembly 108 is insufficiently sensitive to detect changes in distal end bearing over time, an elastic ring positioned between the umbrella and distal socket may be used to increase sensitivity.

While illustrative embodiments have been illustrated and described, it will be appreciated that various changes can be made therein without departing from the spirit and scope of the invention.

1. A locking pin adapter for a lower-limb prosthetic, the locking pin adapter comprising:
a pin lock body configured to receive a locking pin of a liner;
a sensing element having a hollow interior and positioned to receive a portion of the locking pin that extends past the pin lock body; and
sensing circuitry configured to generate a signal indicative of a size of the portion of the locking pin that is within the hollow interior of the sensing element.

2. The locking pin adapter of claim 1, wherein the sensing element includes a wire coil disposed around the hollow interior of the sensing element.

3. The locking pin adapter of claim 2, wherein the circuitry includes a capacitor that forms an LC tank circuit with the coil of the sensing element.

4. The locking pin adapter of claim 3, wherein the circuitry further includes a sensing circuit configured to measure an oscillation frequency of the LC tank circuit.

5. The locking pin adapter of claim 1, further comprising data collection circuitry configured to:
receive the signal from the sensing circuitry; and
transmit collected data for storage on a computer-readable medium.

6. The locking pin adapter of claim 1, further comprising a thermal sensor configured to generate a signal indicative of a temperature of the sensing element.

7. A sensing system for measuring a depth of insertion of a locking pin of a liner in a pin lock of a lower-limb prosthetic, the sensing system including a sensor assembly comprising:
a base;
a sensing element mounted to the base and having a hollow interior; and
a housing that encloses at least the sensing element and includes means for coupling the sensor assembly to the lower-limb prosthetic in a position where the locking pin adapter of the lower-limb prosthetic is aligned with the hollow interior of the sensing element;
wherein the sensing element is configured to generate a signal indicative of a size of a portion of the locking pin that is within the hollow interior of the sensing element.

8. The sensing system of claim 7, wherein the sensing element includes:
a former that creates the hollow interior; and
a wire coil positioned around the former.

9. The sensing system of claim 8, further comprising a capacitor that forms an LC tank circuit with the wire coil.

10. The sensing system of claim 9, further comprising a sensing circuit configured to measure an oscillation frequency of the LC tank circuit, wherein the oscillation frequency is determined in part by the size of the portion of the locking pin that is within the hollow interior of the sensing element.

11. The sensing system of claim 7, wherein the sensing element includes a thermal sensor configured to generate a signal indicative of a temperature of the sensing element.

12. The sensing system of claim 7, further comprising a potting material within the housing.

13. The sensing system of claim 7, wherein the means for coupling the sensor assembly to the lower-limb prosthetic includes a plurality of spacers positioned to transfer load from bolts of a socket of the lower-limb prosthetic to a pyramid adapter of the lower-limb prosthetic.

14. The sensing system of claim 7, further comprising at least one spring positioned distally from the means for coupling the sensor assembly to the lower-limb prosthetic.

15. A method of monitoring residual limb insertion depth in a socket of a lower-limb prosthetic, the method comprising:
receiving, by a computing device, values indicating an oscillation frequency of an LC tank circuit of a sensor assembly; wherein the oscillation frequency is affected in part by a size of a portion of a locking pin present within the sensor assembly after passing through a pin lock body of the lower-limb prosthetic;
determining, by the computing device, an insertion depth value based on the values indicating the oscillation frequency; and
storing, by the computing device, the insertion depth value in a computer-readable medium.

16. The method of claim 15, further comprising:
determining, by the computing device, a physical activity performed by a wearer of the lower-limb prosthetic based on insertion depth values stored in the computer-readable medium.

17. The method of claim 16, wherein the physical activity includes at least one of weight shifting, standing, and walking.

18. The method of claim 15, further comprising receiving, by the computing device, a signal indicating a temperature of the sensor assembly;
wherein the insertion depth value is determined based further on the signal indicating the temperature.

19. The method of claim 15, wherein signal is received from a sensing system including a sensor assembly comprising:
a base;
a sensing element mounted to the base and having a hollow interior; and
a housing that encloses at least the sensing element and includes means for coupling the sensor assembly to the lower-limb prosthetic in a position where a locking pin adapter of the lower-limb prosthetic is aligned with the hollow interior of the sensing element;
wherein the sensing element is configured to generate the signal, which is indicative of a size of a portion of the locking pin that is within the hollow interior of the sensing element.
